(19) United States
(12) Patent Application Publication
(10) Pub. No.: US 2003/0195533 A1
(43) Pub. Date: Oct. 16, 2003

(54) Capsule shaped artificial blood vessel device

(76) Inventor: Jih Chung Chiang, Taichung (TW)

Correspondence Address: CHARLES E. BAXLEY, 5th Floor, 59 John Street, New York, NY, 10038, US

(21) Appl. No.: 10/119,395

(22) Filed: Apr. 11, 2002

(51) Int. Cl. A61B 17/08

(52) U.S. Cl. 606/153

(57) ABSTRACT

An artificial blood vessel device includes capsule having a main part and having a slot formed by two lips and two ends. The lips of the main part of the capsule may be secured to the edges of a groove of a blood vessel, such as the artery, for allowing the blood vessel to be coupled to the capsule without cutting off the blood vessel. The capsule includes two end semi-spherical end parts. One of the end parts of the capsule includes an opening formed by a peripheral flange, for coupling to a vein, and for allowing the vein and the artery to be coupled together.

### BACKGROUND OF THE INVENTION

1. Field of the Invention

The present invention relates to an artificial blood vessel, and more particularly to an artificial blood vessel device having a capsule shape.

2. Description of the Prior Art

Typical artificial blood vessel devices comprise a shape similar to that of the typical blood vessels having a tubular shape and having two ends coupled or secured to the blood vessels after the blood vessels have been cut off. The fibers or the muscular fibers of the blood vessels will be cut and thus will be completely damaged.

In addition, the typical artificial blood vessel devices normally include a diameter similar or close to that of the blood vessels. When it is required to engage a needle of a syringe into the artificial blood vessel device, the users are also required to fist their hands, and a resilient and elastic belt is required to wind around the arms of the users in order to increase the blood pressure in the blood vessels and the artificial blood vessel devices, and in order to expand the blood vessels and the artificial blood vessel devices, for allowing the needle of the syringe to be engaged into the artificial blood vessel device. The engagement of the syringe to the artificial blood vessel device is still difficult.

The artificial vessel is widely used in arteriouvenous shunt construction; i.e., one end of the artificial vessel is coupled on the side of the artery and the other end on the side of the vein. Native vessels are cut longitudinally at one side of its walls during the construction. This is so called graft-bridged arteriovenous shunt medically. Blood flow in the artificial vessel is usually kept more than 300 ml per minute in order to fulfill the requirement of medical treatment. Unfortunately, the artificial vessel usually become occluded when intimae of the native vein become overgrowth or when the user suffered from other systemic disease.

The present invention has arisen to mitigate and/or obviate the afore-described disadvantages of the conventional artificial blood vessel devices.

In addition, this special invention is designed to be used when all conventional methods fail.

### SUMMARY OF THE INVENTION

The primary objective of the present invention is to provide an artificial blood vessel device including a capsule shape for allowing the blood to be easily drawn from the blood vessel and for allowing the other blood material or medicine to be injected or filled into the blood vessel.

The other objective of the present invention is to provide an artificial blood vessel device for attaching onto or for coupling to the blood vessel without cutting off the blood vessel.

The further objective of the present invention is to provide an artificial blood vessel device including a capsule shape for being coupled between the artery and the vein or on the artery only.

In accordance with one aspect of the invention, there is provided an artificial blood vessel device for coupling to a blood vessel having a groove formed by a pair of edges, the artificial blood vessel device comprises a capsule body including a main part having a slot formed therein and defined by two lips and two ends. The lips of the main part of the capsule body are secured to the edges of the blood vessel, for allowing the blood vessel to be coupled to the capsule body without cutting off the blood vessel, and for allowing the blood to be easily drawn from the blood vessel and for allowing the other blood material or medicine to be injected or filled into the blood vessel.

The main part of the capsule body is an intermediate main part having two ends, and the capsule body includes two end parts provided on the ends of the main part.

The end parts of the capsule body each includes a semi-spherical shape. One of the end parts of the capsule body may further include an opening formed therein and defined by a peripheral flange, for coupling to a vein.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is a perspective view illustrating an operation of an artificial blood vessel device in accordance with the present invention;

FIG. 2 is a cross sectional view illustrating the engagement of the artificial blood vessel device as shown in FIG. 1 into the skin of the users, such as into the subcutaneous tissues of the users;

FIG. 3 is a perspective view illustrating the other operation or application of the artificial blood vessel device;

FIG. 4 is a perspective view of the artificial blood vessel device which has a portion cut off for showing the inner structure of the artificial blood vessel device.

FIGS. 5 and 6 are cross sectional views taken along lines 5-5, and 6-6 of FIG. 3 respectively; and

FIG. 7 is a cross sectional view taken along lines 7-7 of FIG. 4, illustrating the engagement of the artificial blood vessel device as shown in FIGS. 3 and 4 into the skin of the users, such as into the subcutaneous tissues of the users.

### DETAILED DESCRIPTION OF THE PREFERRED EMBODIMENT

Referring to the drawings, and initially to FIGS. 1-4, an artificial blood vessel device in accordance with the present invention comprises a capsule body 10 including an intermediate primary or main part 11 having a substantially cylindrical shape, and including two end parts 12 provided on the ends of the main part 11 and each having a substantially semi-spherical shape. The main part 11 includes a slot 14 formed therein, such as formed along a longitudinal direction or axis of the capsule body 10, or formed between the end parts 12, and formed or defined by a pair of lips 15 and two ends 16.

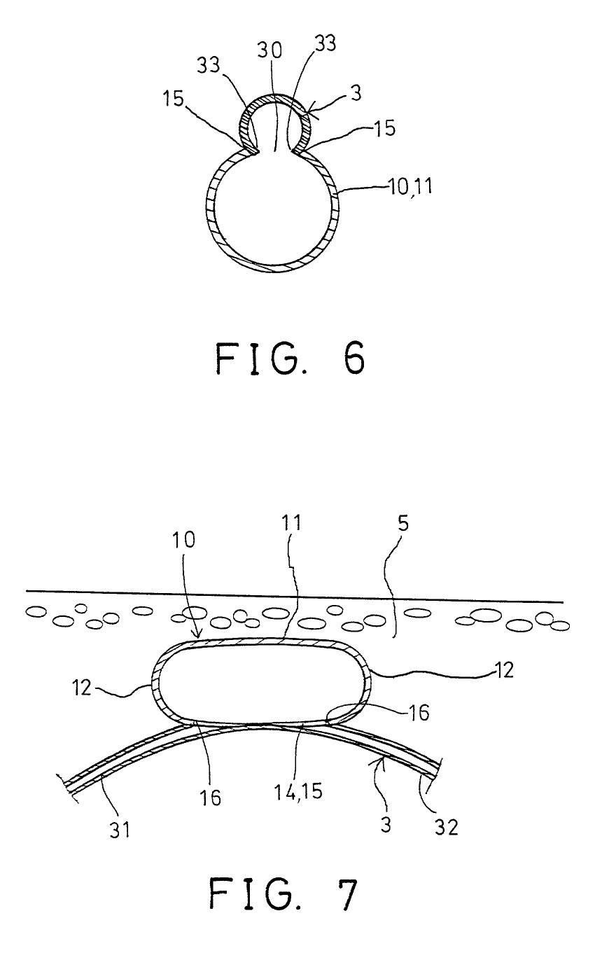

As best shown in FIGS. 1, 3, 5 and 6, the blood vessel, such as the artery 3 is cut with a longitudinal groove 30 therein (FIG. 6), and thus has two edges 33 conforming to the lips 15 of the capsule body 10 and secured to the lips 15 of the capsule body 10 with such as stitches, by such as microsurgery processes. For example, if the capsule body 10 includes a length of about 27 mm, the main part 11 may then include about 20 mm, and the end parts 12 each may thus include a radius of about 3.5 mm. The artery 3 is then also cut to form a 20 mm groove 30 therein in order to conform the lips 15 of the capsule body 10.

As best shown in FIG. 5, the portion of the artery 3 located in the middle part of the capsule body 10 or located between the ends 16 of the slot 14 of the capsule body 10, in operation, will be expanded or flattened, for allowing the artery 3 and the capsule body 10 form a substantially circular shape altogether (FIG. 5). The other portions of the artery 3 located close to the ends 16 of the slot 14 of the capsule body 10 may include a substantially circular or cylindrical shape, or may include a shape closer to the circular or cylindrical shape, as shown in FIG. 6. The blood from the upstream portion 31 of the artery 3 may flow into the capsule body 10 via the slot 14 of the capsule body 10, and may then flow through the downstream portion 32 of the artery 3.

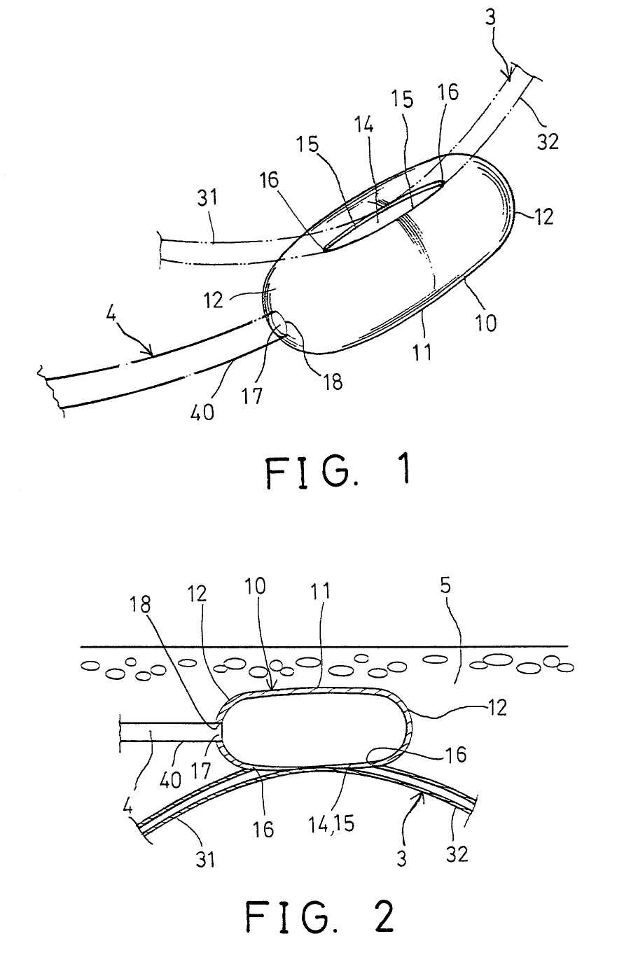

Referring again to FIGS. 1 and 2, the capsule body 10 may further include an opening 17 formed therein and formed in one of the end parts 12 of the capsule body 10, and formed or defined by a peripheral flange 18 for coupling to a vein 4. The vein 4 is cut to form an end portion 40 conforming to the opening 17 and the peripheral flange 18 of the capsule body 10, and secured to the peripheral flange 18 of the capsule body 10 with such as stitches, by such as microsurgery processes, for coupling the artery and the vein together.

In operation, as shown in FIGS. 2 and 7, the capsule body 10 may be engaged into the skin 5, or into the subcutaneous tissues of the users. The capsule body 10 is made of rubber or synthetic materials that include a resilience and/or softness similar to the blood vessels, and that may be recovered after the needle of the syringe has been engaged into and/or removed from the capsule body 10. The doctors or the nurses may easily engage the needle of the syringe into or from the capsule body 10 without increasing the blood pressure in the blood vessels, or without expanding the blood vessels, or without winding the elastic belt around the arms of the users.

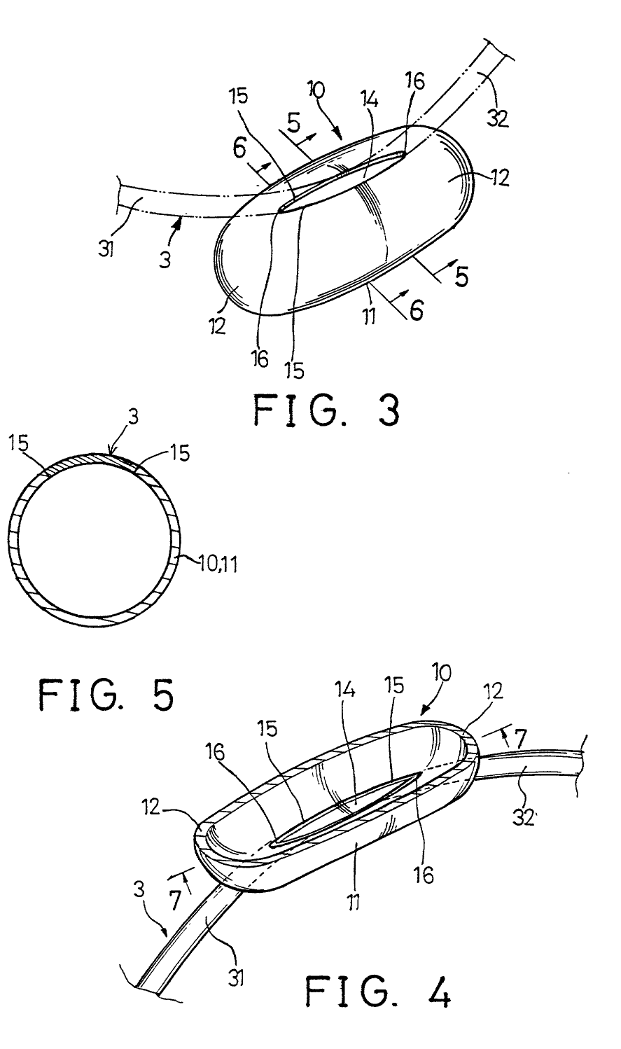

As shown in FIG. 2, after the vein 4 has been coupled to the artery 3 for a period of time, the vein 4 may include a typical inner film or tissue or membrane formed therein for blocking the opening 17 of the capsule body 10, such that the vein 4 is no longer coupled to the capsule body 10, and such that the capsule body 10 may be acted as or may be used as the condition as shown in FIGS. 3, 4, and 7.

Accordingly, the artificial blood vessel device in accordance with the present invention includes a capsule shape for allowing the blood to be easily drawn from the blood vessel and for allowing the other blood material or medicine to be injected or filled into the blood vessel, and/or for attaching onto or for coupling to the blood vessel without cutting off the blood vessel, and/or for being coupled between the artery and the vein.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. An artificial blood vessel device for coupling to a blood vessel having a groove formed by a pair of edges, said artificial blood vessel device comprising:
a capsule body including a main part having a slot formed therein and defined by two lips and two ends,
said lips of said main part of said capsule body being secured to the edges of the blood vessel, for allowing the blood vessel to be coupled to said capsule body without cutting off the blood vessel.

2. The artificial blood vessel device according to claim 1, wherein said main part of said capsule body is an intermediate main part having two ends, and said capsule body includes two end parts provided on said ends of said main part.

3. The artificial blood vessel device according to claim 2, wherein said end parts of said capsule body each includes a semi-spherical shape.

4. The artificial blood vessel device according to claim 2, wherein a first of said end parts of said capsule body includes an opening formed therein and defined by a peripheral flange, for coupling to a vein.
